(19) United States
(12) Patent Application Publication
(10) Pub. No.: US 2012/0232350 A1
(43) Pub. Date: Sep. 13, 2012

(54) RETRACTOR BLADE INCLUDING A FLEXIBLE MEMBER FOR ANCHORAGE ENGAGEMENT

(76) Inventor: Kevin Seex, Kingswood (AU)

(21) Appl. No.: 13/393,318

(22) PCT Filed: Aug. 31, 2010
(86) PCT No.: PCT/AU2010/001116

§ 371 Date: May 21, 2012

(30) Foreign Application Priority Data
Aug. 31, 2009 (AU) 2009904143

(51) Int. Cl. A61B 1/32

(52) U.S. Cl. 600/206; 600/213

(57) ABSTRACT

A retractor blade for retracting soft tissues during surgery, the blade having a blade body having a first proximal end and a second distal end, the first end including a structure to allow connection of the blade body to a blade support member; a formation at its distal end which is capable of releasably engaging a second co-operating retractor blade; and a structure to engage the retractor blade to an anchor.

### BACKGROUND

The present invention relates to retraction assemblies used in surgery, for retracting soft tissue particularly though not exclusively in spinal surgery. The invention further relates to a retracting blade having a flexible retaining thread capable of releasable engagement with a bone anchor and/or co operating retractor blade to secure the retractor blade for surgery.

The invention further provides a retraction blade having a flexible wire, thread or the like which co operates with a retractor blade to enable engagement of an anchorage fastener after the fastener has been set in bone. The invention further relates to a retractor blade which includes closed or open through passages or channels which provide a track for the flexible thread or wire to enable remote access to the thread or wire so the retractor may be secured or released from engagement with an anchorage by adjustment or removal of the thread.

The invention further relates to a retractor blade assembly including opposing retractor blades each of which include through passages to receive a co operating retaining thread which allows the blades to co operate in retraction by engagement with and disengagement from an anchor by loading or unloading the thread.

### PRIOR ART

There are in existence a number of assemblies used in retraction of soft tissues and which include retractor blades which engage bone during such retraction. In particular, there are known retractor blades which retract soft tissue during spinal surgery. Such retractor blades are maintained at a setting to allow a surgeon access to a spinal disc space and vertebrae in cervical and lumbar spinal disc surgery.

Retractors are sometimes used in conjunction with distraction assemblies. The process of separating vertebral bones is intervertebral distraction. This involves insertion of a spreading type instrument into an excised disc space which engages upper and lower vertebral bodies and when applied, separates them. Alternatively, in the cervical spine in a procedure known as non-intervertebral distraction may be used in which long screws are inserted into the upper and lower vertebral bodies. Surrounding soft tissues must be held apart by retractors. Once retracted, there is a natural elastic recoil of the stretched tissues so it is essential to employ retractors which effectively restrain soft tissues and without loosing the requisite retraction.

It is important to reduce trauma to soft tissues which may occur from contact with parts of retractor blades so that the surgical procedure is as minimally invasive as possible and thus minimally traumatizes the organs, tissues and vasculature being displaced to allow access to the vertebral region being treated. Various types of retractors are known each having means to enable retention by a support member.

Anterior lumbar surgery can be performed for a number of reasons, but most commonly this is for excision of degenerate intervertebral disc after which a fusion procedure or lumbar disc arthroplasty is performed. Amongst spinal surgeons it is recognized that the most difficult and dangerous part of the surgery on the anterior lumbar disc spaces is dissection, mobilization and maintenance of retraction of the vessels, and in particular the left common iliac vein. Whatever level of surgery is being performed, there is an initial approach requiring some degree of vascular dissection. The approach and surgery generally requires the use of handheld retractors, at least initially, which may then be replaced with fixed retractors to maintain retraction for the rest of the procedure.

Fixed retractors require either internal fixation to spinal vertebra or external fixation using a table mounted system. Retractors are usually positioned to hold tissues away from the surgical field both laterally (side to side) and longitudinally (up and down) relative to a spine.

Existing retractors may be internally or externally fixed. Internal fixation of retractors is utilized to hold the left common iliac vein or other tissues in a retracted position to avoid the danger of puncturing or squeezing of veins. Due to the difficulty and dangers of moving and keeping the blood vessels retracted during anterior lumbar surgery, stability of the retractors is particularly important. The most stable retractors are those embedded in the bone e.g. Steinman pins and Hohmann retractors. Steinman pins are long pins impacted into the bone while Hohmann retractors are conventional retractor blades with a curved pointed end which can be impacted into the bone for stability. Some limited movement of the Hohmann blade is possible by bending. Also known are standard retractor blades that have a through passage which allows separate introduction of sharp pins through the passage into the vertebral body thus securing the blade to the vertebra. It is lateral as distinct from longitudinal retraction that represents the greatest difficulty as the soft tissues will naturally want to return to their normal anatomical location by migrating under a distal end of the blade which would normally be in engagement with vertebral bone.

Internally fixed retractors have limitations. Although very stable once in place they are not adjustable and insertion does produce bone injury. Insertion and removal can also be hazardous to vessels or other soft tissues. The known externally fixed retractor blades have significant limitations.

Contact and pressure on the blade against the bone improves stability of retractor blades. Most conventional blades however rely only on their external fixation for stability. If there is contact with the bone it lies at the side of the vertebral body usually at or above the equator of the vertebrae where the bone is curving postero-laterally.

To remain stable they the blades rely on a solid immobile connection to the operating table through various linkages and if in contact with the bone, a force is directed along the edge of the blade (the lip) parallel to this radius of the body. Many conventional blades just sit beside the bone with a lip curving away from the bone.

When a patient's spine moves e.g. during impaction or positioning of implants, or other vigorous work, the lateral retractors tend to bounce and slip. In that case, soft tissues including vessels can slip under or around the retractor blades.

Contact with the bone by providing an additional point of stability helps reduce this, but it remains a problem. If a constant force is applied from the frame along the line of the blade, pushing the blade against the spine helps stability, but this can easily lead to the blade slipping or sliding posteriorly and causing tissue injury when the spine moves. Inadvertent downward pressure by surgeons or their assistants on these blades also is a problem as it leads to posterior displacement, because there is usually little or no support for the blade from the bone relying as it dies essentially on friction grip. This can lead to soft tissue injury.

By comparison when retracting longitudinally using retractor blades, the edge of the blades can be pushed down onto the anterior surface of the vertebral bodies where they are lying across the spine and perpendicular to the main radius. This gives them much greater relative stability. The stability is also enhanced because the anterior surface of the spine is less curved anteriorly than laterally.

In addition to instability leading to tissue injury, it is also preferable to improve stability as this reduces the operating time lost spent adjusting retractors. In general terms the morbidity of surgery tends to increase with increasing length of procedure so if the surgical procedure time can be reduced by more efficient retraction it follows that morbidity rate can decrease.

One of the limitations of many existing retraction systems is the tendency for the vessels, in particular the left common iliac vein, to bulge around the retractor, which can expose the vein to injury and impede the surgeon.

Numerous retractor blades exist for use in surgery of various shapes and geometry. Although there are a wide variety of retractor blades currently in use, in view of the disadvantages inherent in those blades, there remains room for improvement in the operation, anchorage of retractor blades with the objective of providing a blade which is easy to use, adaptable to existing support frames, efficiently maintains retraction of soft tissue and does not obstruct the surgeons path to the operating site, can be installed and released conveniently and does not compromise retraction in the event of unwanted loading such as inadvertent bumping during surgery.

One solution that has been provided to this problem is a co operating cannulated retractor blade which receives an anchor pin or screw, which is screwed into bone during surgery. These pins apply a downward force and secure the blade against the bone by means of mutual engagement between the cannulation in the blade and the anchor pin. This provides good anchorage but there are dangers inherent in finding the optimal pin position and holding the blade in a position near the optimal pin anchorage position until the pin is passed through the blade. There is a danger to anatomical structures when the pin is moved around to find the optimal anchorage position as the pin could snag vital structures such as blood vessels. There is another disadvantage of the prior art assemblies in that the surgeon must hold the retractor blade perfectly still in the critical selected position while sliding a sharp potentially dangerous pin through the cannulation in the blade and into engagement with the spine. Moving the retractor blade while the pin is on the bone incurs the risk of dragging the point of the pin over a difficult to see compressed vein which could be inadvertently impaled during pin insertion. An error of this kind can have fatal consequences. Correct or optimal placement of the retractor blade is critical to its performance The angle at which the blade sits relative to the spine is critical to its performance as is the angle of entry of the pin.

There has been a recent growth in interest in new ways of reducing the amount of injury to the soft tissues when performing spine surgery. New operations, equipment and approaches have been developed for this purpose and often labelled ‘minimally invasive’. Conventional self retaining spinal retractors, unless table mounted, are fixed in position and held stable by tissue counter pressure. They exert pressure on the tissues constantly which is only relieved by removal of the instrument.

An earlier patent application by the present applicant teaches systems in which a retractor blade can rotate allowing variable retraction and tissue relaxation. This intermittent release of retraction has been shown to be of benefit in reducing muscle damage and post operative pain. Rotation of retractor blades is generally desirable as it allows tissue retraction when required and tissue relaxation otherwise. Rotation about an axis in the wound is particularly suited to the spine where the surgical targets are well defined and the position of the retractors is relatively constant allowing retraction for the procedure to be achieved from one fixed axis position. In order to provide a stable axis of rotation inside the wound some method of anchorage in the wound is required.

The applicant has also filed an earlier patent application which describes a retractor blade assembly and method of use by which retractors can be reversibly secured to the patients bone or soft tissues allowing for stable retractor rotation during surgery.

Suture anchors are known see for example (U.S. Pat. No. 5,584,860) and are used for the purpose of repair of tendons by providing a bony anchorage to which the suture and injured tendon can be fixed. As far as the applicant is aware, these suture anchors have not been previously described as a means of anchoring retractors, and they are designed for permanent implantation.

Cannulated retractors are well are known. One example of a type of cannulated blade with a curved surface has been described by the present applicant in a recently filed application under the Patent Co operation Treaty—application No. PCT/AU2010/000431. Other than insertion of a rigid anchorage pin into the cannulations in such retractors, it is not known in the prior art to employ a flexible thread fed through such cannulations for the purpose of anchoring a retractor.

US application 20070106123 (“Serengeti Retractor”) discloses a flexible polymer retractor with two arcuate arms which is placed around a screw during percutaneous screw insertion. Spreading the arms provides greater wound retraction which is helpful for subsequent steps including rod and cap insertion. The flexibility provides an advantage over stiff metal retractors which are secured to the top of the pedicle screw but create a fixed narrow tunnel of view down to the screw head.

There are some clinical limitations of the Serengeti retractor. It has to be inserted, attached around the shank and under the screw head. The retractor cannot be placed after screw insertion. Thus if one side of the device breaks during screw insertion (because of compression under the screw head) the screw has to be removed completely to remove and replace the device. This is undesirable as the bone purchase may be reduced by a second insertion. Another limitation is that to remove the device, it requires to be fractured in two places to be removed easily, this requires use of a special tool. During removal the retractor sometimes fractures on one side only, making removal, time consuming and fiddly. A third clinical limitation which occurs because they cannot be inserted after the screw and rod is that they cannot be used in revision cases prior to screw and rod removal.

Handheld pedicle screw retractors are known with a shape at the distal portion of the blade in the form of a semicircle that engages the screw shank under the head of a pedicle screw. These handheld retractors are bulky and have to be continuously held to maintain stability and prevent them falling out. They do however engage against what in effect is a bony anchor.

There remains a need to constantly provide a more convenient and safer means of installation of retractors and also a more efficient means for engagement and disengagement between an anchor and retractor blade, which ameliorates or eliminates the aforesaid disadvantages of the prior art.

### INVENTION

The present invention addresses the problems associated with the known retraction blades and seeks to provide an alternative to known retractor systems by providing a retractor blade including a retaining thread which allows releasable anchorage of a blade to an anchor pin or to another blade.

The present invention provides retraction assemblies used in surgery, for retracting soft tissue particularly though not exclusively in spinal surgery, and comprising a retracting blade which has a through passage which receives a retaining thread to detachably receive an anchorage fastener once the fastener has been set in vertebral bone or to engage and co operate with an opposing thread.

Although the invention is described below in relation to the anterior lumbar spine, the principles and geometry embodied in the retractor blade have applications throughout the spine and wherever bone is available to provide support.

Threading a flexible line through this single cannulated blade for the purpose of securing flexibly to an anchor has not been described. Use of a single channel or cannulation is less functional than the device described in the present application here because the shape and disposition of the distal loop is uncontrolled from a single opening. In addition if pulling a distal loop tight through a single channel, should the loop disengage from the anchor, the line will be pulled out of its channel and the blade will require rethreading. Threading the distal loop line through a separate member to prevent line withdrawal accidentally would avoid this, but removal of the assembly is less functional as the line cannot be cut and pulled through without the separate member falling off in the wound. With this separate member assembly, the distal loop and member can only be unhooked to conveniently remove device.

The retractor according to the invention seeks to improve the installation and removal of a blade from an anchor pin during an operation such as anterior lumbar surgery. Although designed to aid retraction during surgery on the anterior lumbar spine, the principles of the invention have application in other spinal locations and elsewhere where bone is available for load distribution.

In its broadest form the present invention comprises:

a retractor blade for retracting soft tissues during surgery, the blade comprising:

a blade body having first and second ends, the first end providing means to allow connection of the blade body to a support member,

the blade further comprising a retaining thread which is fed through at least one opening in the blade to allow releasable engagement of the second end of the blade to a bone anchor and/or to an opposing retractor blade via the retaining thread.

According to a preferred embodiment, the at least one opening comprises a series of aligned passages through which the retaining thread is threaded. Preferably free ends of the thread extend from the first (proximal) end to allows the surgeon to control the action of the threads for the purpose of engagement and disengagement.

Although an optimal arrangement for the openings are aligned elongated tubular passages, to receive the thread, it will be appreciated by persons skilled in the art that alternative passages and openings are feasible depending upon the nature and extent required of the looping of the thread to engage an anchor.

According to one embodiment, the passages in the retractor blade are disposed in alignment along a longitudinal axis of a blade. Alternatively, the passages are disposed along side edges of the blade. According to an alternative embodiment, the passages pass at least part way along the length of a blade. Alternatively the passages may be an array of small openings.

In another broad form the present invention comprises:

a retractor blade for retraction of soft tissue, the blade comprising: a generally elongated blade body having first and second ends, the first end providing means for connection of the blade body to a support member, the second end comprising means to enable anchorage of the blade,

characterised in that said means to connect the blade body at its distal end comprises a flexible thread capable of forming a connecting loop at or near the second end to enable releasable engagement of the thread with a bone anchor.

In another broad form the present invention comprises:

a surgical retractor blade for retraction of soft tissue the blade comprising: a generally elongated blade body having first and second ends, the first end providing means for connection of the blade body to a support member, the second end including means to enable the retractor to engage an anchor via a co operating retaining thread.

According to the preferred embodiment, the engaging means includes at least one opening in the blade which receives the retaining thread. Preferably the retaining thread is one piece and is adapted to form a retaining loop at the second (distal) end of the blade which is capable of engaging a bone anchor or a corresponding distal end of a co operating retractor blade.

According to a preferred embodiment the retaining thread passes through aligned passages. According to one embodiment the passages are cannulated and may be disposed along an edge or a face of each retractor. The aforesaid retractor blade is intended to meet the objectives of optimal stability and safe and efficient retraction of soft tissue.

In another broad form the present invention comprises:

a retractor blade assembly for retraction of soft tissue during surgery, the blade assembly comprising: at least one generally elongated blade each having first and second ends, the first end providing means for connection of the blade to a support member, the second end including a thread which co operates with the blade and engages an anchor to secure the at least one blade in a predetermined location.

Preferably the thread exits a first opening in the second end of the blade and enters a second opening in the second end to define a closed loop. The free ends of the thread exit the blade via openings in the proximal end of the blade thereby enabling a surgeon tension the closed loop.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims.

### BRIEF DESCRIPTION OF THE DRAWINGS

The present invention will now be described in more detail according to a preferred embodiment but non limiting embodiment and with reference to the accompanying illustrations, wherein:

FIG. 1 shows an elevation view of a blade including aligned passages for a retaining thread according to one embodiment.

FIG. 2 shows the blade of FIG. 1 including a retaining thread.

FIG. 3 shows the blade of FIG. 1 abbreviated with a distal retaining an anchor via a loop of retaining thread.

FIG. 4 shows an elevation view of a blade including lateral aligned passages with a retaining thread forming an outside loop at a distal end according to an alternative embodiment.

FIG. 5 shows the blade of FIG. 4 with the retaining thread forming a loop with alternative geometry.

FIG. 6 shows a top view of the blade of FIG. 1.

FIG. 7 shows an enlarged view of the distal end of the blade of FIG. 4 indicating the path of the retaining thread.

FIG. 8 shows an anchor screw engaged via the retaining thread at a distal end of an abbreviated retractor blade.

FIG. 9 shows an enlarged view of a distal end of an abbreviated retractor blade of the type in FIG. 4 indicating an alternative path for the retaining thread.

FIG. 10 shows an assembly of opposing retractor blades according to an alternative embodiment and including a retaining thread engaging the distal end of each blade through an array of apertures.

FIG. 11 shows a screw anchor according to one embodiment with retaining hook adapted to receive a retaining thread.

FIG. 12 shows a screw anchor according to an alternative embodiment with retaining hook adapted to receive a retaining thread

FIG. 13 shows an assembly of opposing retractor blades according to an alternative embodiment and including a retaining thread engaging the distal end of each blade through a passage formed in each blade.

FIG. 14 an assembly of opposing flat side retractor blades according to an alternative embodiment and including a retaining thread engaging the distal end of each blade through a passage formed in each blade.

FIG. 15 shows a retractor with cannulated legs in a resting state and openings which receive in loose weaving therethrough a retaining thread.

FIG. 16 shows a retractor similar to that shown in FIG. 21 with retaining thread drawn tight to form a retaining loop.

FIG. 17 shows a blade where distal loop emerges a distance from the end, for use in revision cases where a rod joining two or more screws is already present preventing the loop getting underneath the screw head.

FIG. 18 shows with corresponding numbering an enlarged view of the lower component of FIG. 17.

FIG. 19 shows a perspective exploded view of a blade assembly including a pair of opposing blades.

FIG. 20 shows an enlarged perspective view of a lower portion of a retractor blade according to an alternative embodiment.

FIG. 21 shows a pair of co operating retractor blades.

### DETAILED DESCRIPTION

This description will concentrate on a single bladed retractor for ease of understanding but the principles apply equally to multiple blades and variable number and types of anchors which receive the retractors.

FIG. 1 shows a front perspective view of a retractor blade 1 according to a preferred embodiment. The blade to be described below according to its various embodiments, is capable of adaption to various retraction assemblies. Blade 1 comprises: a generally elongated blade body 2 having first end 3 and second end 4. First (proximal) end 3 is connectable to a blade support assembly (not shown). Second (distal) end 4 terminates in an edge 5 at least part of which includes a curved recess 6.

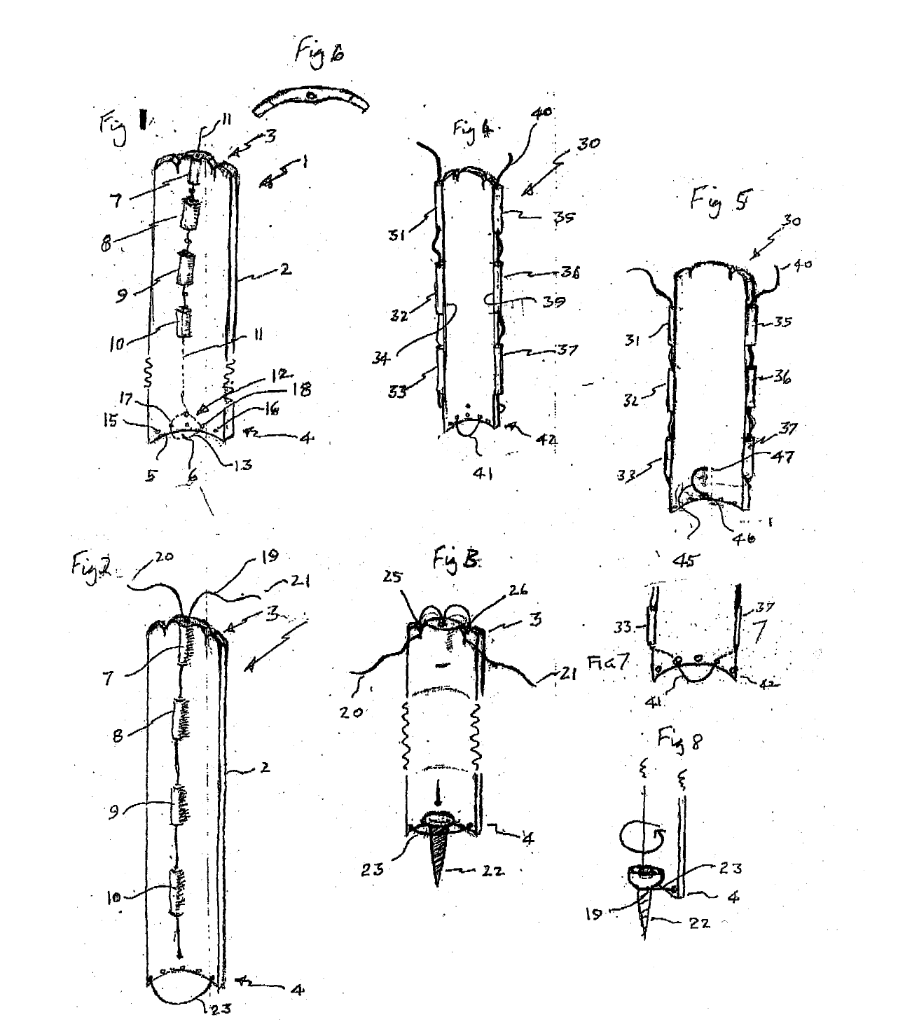

Retractor 1 further comprises an array of aligned longitudinal passages 7, 8, 9 and 10 which provide a path for retaining a flexible thread or wire 11. (see FIG. 2). The passages 7, 8, 9 and 10 can be provided in the form of cannulations with openings to receive thread 10. Distal end 4 of blade 1 comprises an array of holes 12 through which wire 11 may be selectively fed. A distal loop 13 may be formed by thread 14 which is connected to wire 11. Threaded loop 13 may be altered in size depending upon which holes are selected for feeding the thread. For example a larger loop can be obtained by feeding thread 11 through holes 15 and 16 than that which could be formed by feeding the thread 11 through holes 17 and 18. The thread material is preferably a flexible strong material such as but not limited to nylon.

FIG. 2 shows the blade 1 of FIG. 1 with corresponding numbering and including a retaining thread 19, threaded through openings 7, 8, 9 and 10. Thread 19 is preferably a single strand fed through the openings so that free ends 20 and 21 extend from proximal end 3.

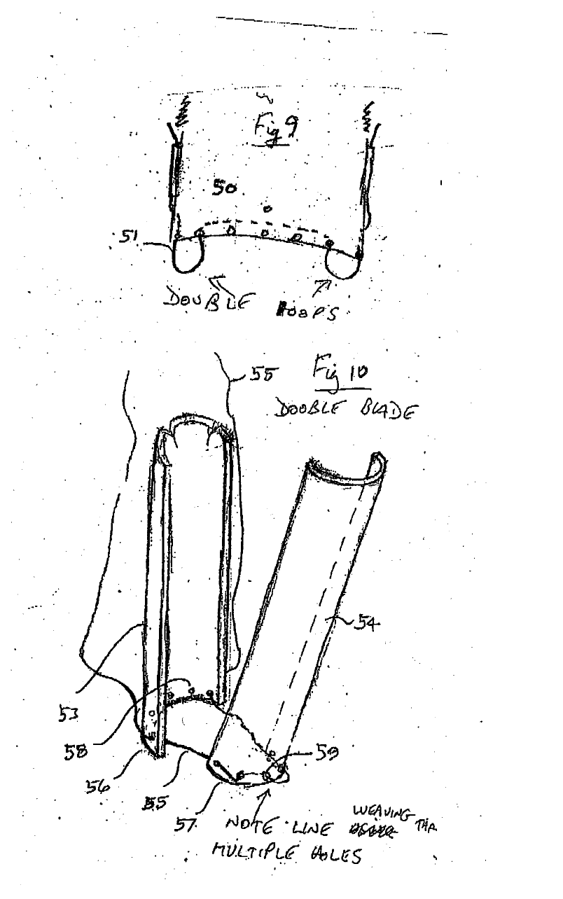

FIG. 3 shows the blade 1 of FIG. 1 abbreviated with a distal retaining an anchor 22 via a distal loop 23 of retaining thread 19. Thread 19 terminates in free ends 20 and 21 which may be urged into notches 25 and 26 to lock thread 19 against slippage which could loosen engagement about anchor 22. Securing notches 25 and 26 at the top of the blade may be added to permanently secure ends of the thread 19, while allowing the free end to be releasably engaged in order to vary the line tension as required.

FIG. 4 shows a retractor blade 30 with lateral aligned passages with a retaining thread forming an outside loop at a distal end according to an alternative embodiment. Blade 30 includes a first array of cannulations 31, 32 and 33 along edge 34 and a second array of cannulated passages 35, 36, 37 and 38 along edge 39. Threaded through these passages is thread 40 which defines loop 41 at distal end 42. Thread 40 terminates in free ends 43 and 44 which can be pulled by the surgeon to tighten loop 41 about an anchor (as in FIG. 3).

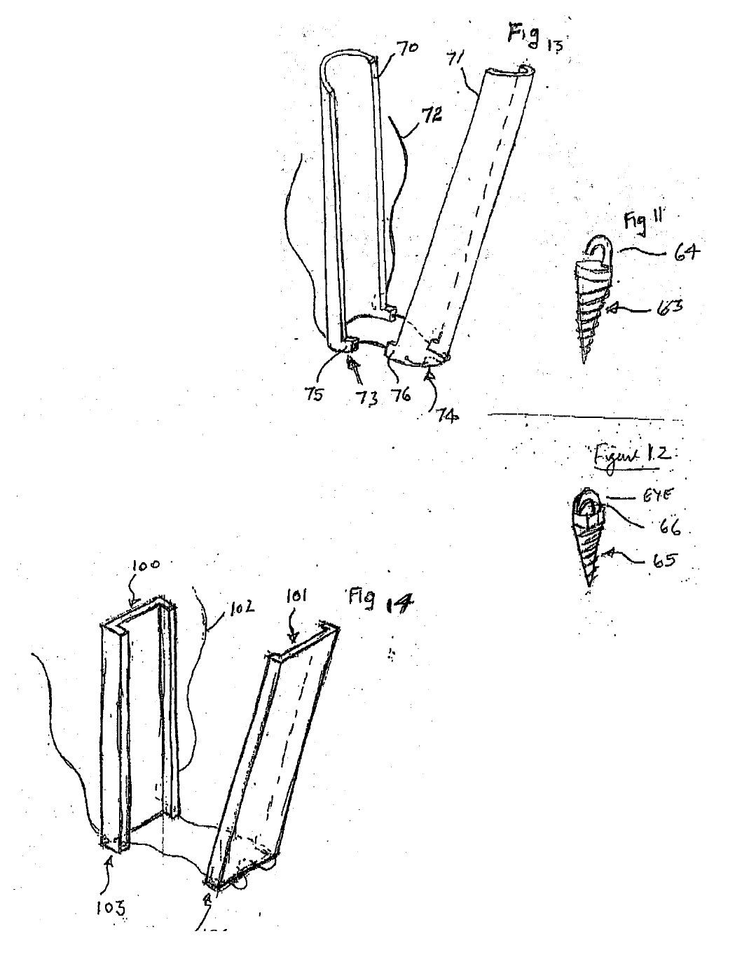

FIG. 5 shows the blade 30 of FIG. 4 with the retaining thread 40 forming a loop 45 with alternative geometry created by feeding thread 40 through vertically aligned openings 46 and 47. Orientation and spacing of distal holes in the blade allow the nylon loop 41 to be horizontal (FIG. 4) or a vertical loop 45 disposed (FIG. 5). The application to engage around a vertically disposed screw head would tend to use horizontal hoop 41 while attachment to a more horizontal anchor would be best done with a vertical hoop 45. Reduced Hole spacing creates a narrow loop useful to engage anchors requiring a tighter fit—see FIG. 4 and FIG. 2.

FIG. 6 shows a top or cress sectional view of the blade of FIG. 1.

FIG. 7 shows with corresponding numbering an enlarged view of the distal end of the blade 30 of FIG. 4 indicating the path of the retaining thread 40.

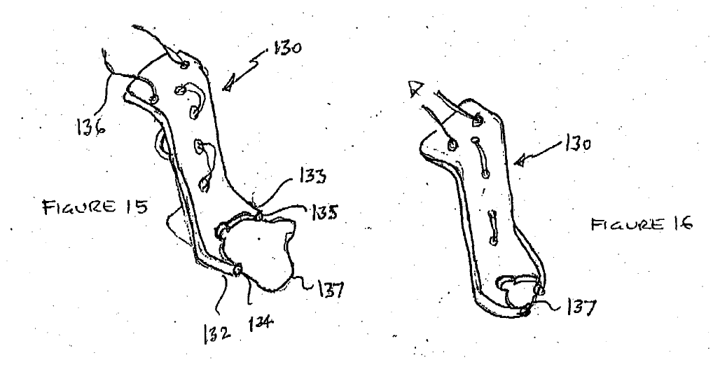

FIG. 8 shows anchor screw 22 engaged via loop 23 of the retaining thread 19 at a distal end of abbreviated retractor blade 1. Distal loop 23 is used to engage around 22 anchor within a surgical wound. Tightening thread 19 draws loop 23 more tightly around the anchor 22 as shown in FIG. 8. Blade 1 is drawn close to the anchor 22, with the distal end 4 finishing close to the anchor 22. Securing the proximal end of the thread 19 to the proximal end of the blade 1, under tension, will prevent thread 19 sliding and maintain the blade adjacent to the anchor. The flexibility of the thread 19 will retain the ability to move flexibly dependent on the tension applied and the properties such as elasticity of the thread 19. Blade 1 is still able to rotate axially around the anchor to its optimal position. The relative fixation to the anchor also allows rotation of the proximal blade end 3 moving relative to the anchored distal end 4 for retraction or relaxation of the soft tissues. Removal of the blade requires loosening the tension in thread 19 to ‘unhook’ it from the anchor 22 and allow the assembly to be withdrawn intact from the wound. Alternatively the thread 19 can be cut anywhere and the thread 19 and blade 1 withdrawn. No special tools are required for removal. One major advantage of this system is that the loop 23 will secure around any prominent shape, anatomical feature or implanted anchor be it a screw, a strong clip that grips bone or other anchoring device. Preferably the anchor is featured to easily secure the thread. Examples include most screws including those that have screw head wider than the screw shank, the shank providing a neck for retaining the loop 23. The distal loop can also be secured to the deep soft tissues with an absorbable anchoring suture, allowing removal of blade and line but leaving the anchor suture. Hereafter any reference to an anchor may include any of the aforementioned anchor types.

Cannulation(s) or passages in the blade may be added to provide smooth and protected passage of the thread to and from the proximal end. These may be fenestrated to allow the easier threading of flexible materials and bending of the retractor blade if made of a flexible material (see FIGS. 1 and 4). Grooves or other channels may also be used around corners or irregular blade features to allow smooth passage of the thread. Holes in the blade may also be used to weave the line and control passage of line this way (see FIGS. 2, 4, and 5).

FIG. 9 shows an enlarged view of a distal end of an abbreviated retractor blade 50 of the type in FIG. 4 indicating an alternative path for the retaining thread 51.

The retractor blades described above can work independently by attachment of the thread to an anchor. Alternatively, one blade can work in co operation with another blade as will be described below.

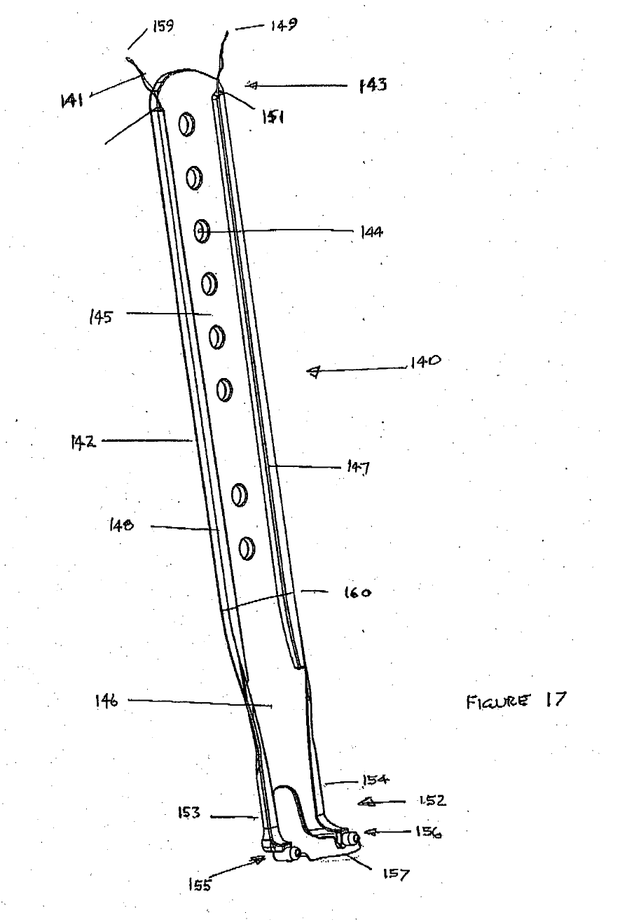

FIG. 10 shows an assembly of opposing retractor blades 53 and 54 according to an alternative embodiment and including a retaining thread 55 engaging the respective distal ends 56 and 57 of each blade through an array of apertures 58 and 59. FIG. 11 shows a screw anchor 63 according to one embodiment with retaining hook 64 adapted to receive a retaining thread.

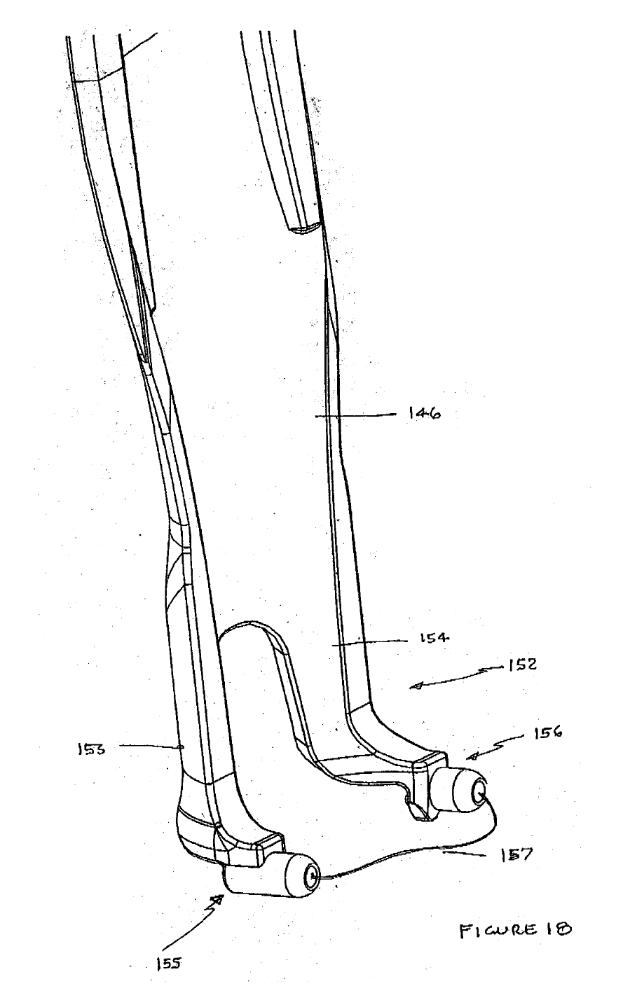

FIG. 12 shows a screw anchor 65 according to an alternative embodiment with retaining hook 66 adapted to receive a retaining thread

In addition to the distal openings, the distal portion of the blade includes formations which allow two or more blades to engage together at their distal ends forming shapes eg legs. These features may also be cannulated for the threaded line so that tightening of a distal loop of thread guides these formations to engage the matching feature in a stable manner and tightening may secure this engagement. FIG. 13 shows an assembly of opposing retractor blades 70 and 71 according to an alternative embodiment and including a retaining thread 72 engaging the respective distal ends 73 and 74 of each blade 70 and 71. Blades 70 and 71 includes respective formations 75 and 76 through which thread 72 travels.

FIG. 14 an assembly of opposing flat side retractor blades 100 And 101 according to an alternative embodiment and including a retaining thread 102 engaging the distal ends 103 and 104 of each blade through a passage formed in each blade.

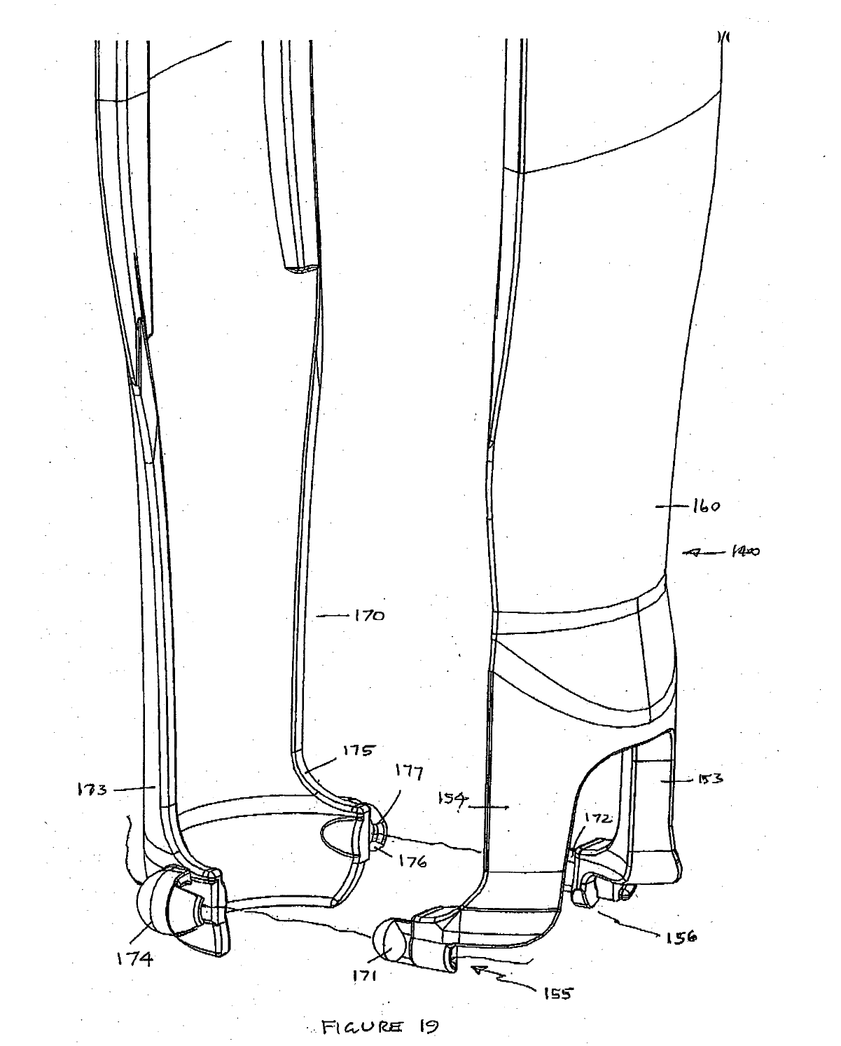

FIG. 15 shows a retractor 130 of FIG. 20 with cannulated legs 132 and 133 in a resting state and openings 134 and 135 which receive in loose weaving therethrough a retaining thread 136.

FIG. 16 shows retractor 130 of FIG. 21 with retaining thread 136 drawn tight to form a retaining loop 137.

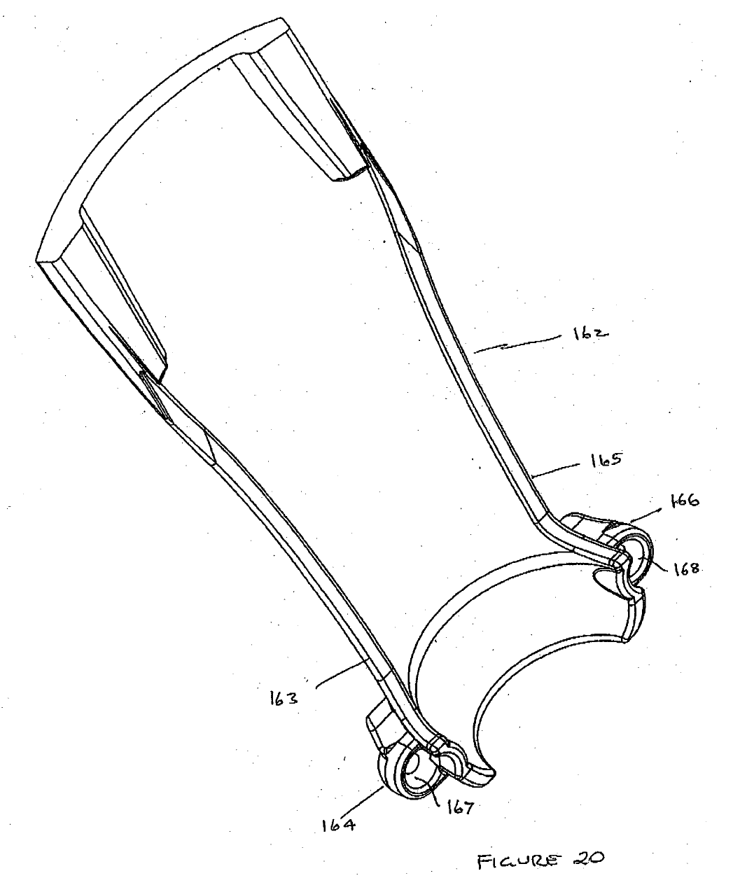

FIG. 17 shows a perspective view of a retractor blade 140 which is capable of use on its own with a co operating one piece thread 141 or in co operation with a corresponding retractor blade. The latter arrangement will be described in more detail below. Blade 140 comprises a blade body 142 having a proximal end 143 including openings 144 which selectively and optionally engages a support member (not shown). According to the embodiment shown blade body 142 has an upper component 145 and lower component 146 which are separable. Upper component 145 includes lateral formations 147 and 148 which receive thread 141. End 149 of thread 141 enters opening 140 in lateral formation 147 and exits from opening 151 in formation 148. This leaves opposite free end 159 of thread 141 extending from formation 148. Upper component 145 is capable of separation from lower component 146 at region 160. Lower component 146 terminates at its distal end 152 in an end profile which includes legs 153 and 154 which each terminate in respective profile parts 155 and 156

Thread 141 emerges from profile parts 155 and 156 to form a loop 157 which is intermediate free ends 149 and 159 of thread 141. A retractor blade of this type is suitable for use in revision cases where a rod joining two or more screws is already present preventing loop 157 getting underneath the screw head. FIG. 18 shows with corresponding numbering an enlarged view of the lower component 146 of FIG. 17.

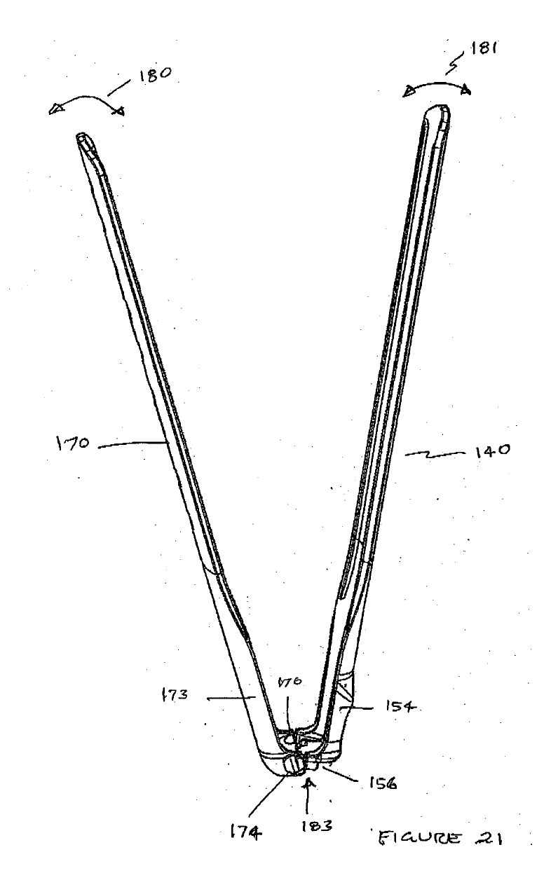

FIG. 19 shows an enlarged perspective exploded view of a blade assembly including a pair of opposing retractor blades 140 and 170. Formation 155 includes male profile part 171 and formation 156 includes male profile part 172. Leg 173 of retraction blade 170 terminates in formation 174 and leg 175 terminates in formation 176. Female profile part of formation 174 engages corresponding male profile part 171. Likewise female profile part 177 engages male profile part 172.

FIG. 20 shows an enlarged perspective view of a lower portion of a retractor blade 162 according to an alternative embodiment. Blade 162 adopts the function of blade 170 in FIG. 19 but with alternative mating geometry.

Leg 163 of retraction blade 162 terminates in formation 164 and leg 165 terminates in formation 166. Female profile part 167 of formation 164 engages a corresponding male profile part as described in FIG. 19. Likewise female profile part 168 engages a corresponding male profile part as described in FIG. 19.

As shown in FIG. 21, retractor blade 140 may co operate with another opposing blade 170 with a point of union of two opposing like retractor blades located at a predetermined but variable, distance from a base. Blades 140 and 170 are capable of mutual rotation and mutual engagement and disengagement via ‘hinge’ 183. Blade 170 rotates in the direction of arrow 180 and blade 140 rotates in the direction of arrow 181.

The clinical application for this embodiment of co operating blades is in a revision fusion case where pedicle screws and rods are already in situ. For example two opposing blades 140 and 170 can be inserted over a pedicle screw head and around the rod with the distal loop and/or opposing features of the blades making contact with each other above the level of the in situ rod (not shown). The distal edge 143 of the blade 140 would still engage the bone giving it some stability while the loop would engage the screw head above the rod. Preventing the blades from separating. Outward rotation of the blades could still occur with rotation or bending occurring with its axis at the level of the loop 141. This would have application in a revision case as described in limitations of the Serengeti retractor mentioned above.

In another embodiment, the two free ends of the thread are joined by a separate member eg clip. This clip then can be pulled to tighten line. Clip is then secured in some way to keep tension in the line. Numerous other ways of securing one or both of the thread ends are envisaged.

The preferred features of the blades described are the at least two distal holes to allow threading of the thread close to the anchor. The blade itself may be curved and tapered to enable smooth insertion but also can be a conventional flat shape, curved in any plane or any combination as desired. Another non limiting example is a blade with features like a male and female connection creating a very firm and locked engagement. Alternative engagements of features may allow movement in certain planes for blade rotation. Narrow legs or protrusions may also be shaped to engage favorably under the anchors head increasing purchase.

There are several circumstances where a flexible material eg polymer would be an advantage and is envisaged. Flexible materials for the blade are also envisaged to allow bending of the blade at any desired point or area in the blade. This bending could be to increase retraction about a fixed distal end. Narrower sections may be formed ‘living hinges’. Also in drawing the loop tightly, a flexible material may be drawn like a draw string snug around the anchor's neck. This can be further enhanced if distal blade has cannulated legs which will conform better to the neck of the securing anchor increasing its purchase. (see FIGS. 21, 22). Others circumstances are envisaged where a more rigid metal blade would be required for the strength without the bulk.

One or more blades can be threaded together creating a device with 1 or more blades that can be separated widely according to the length of the line but then also drawn tightly together for easy insertion. Such a retractor has the advantages of a tubular system with minimal size for insertion. By loosening the line the blades would then be able to move outwards to increase the exposure in any direction.

Pedicle screw retractors lack stability in the wound and can be improved by the addition of the holes and thread as described above to improve stability in the wound (see FIGS. 21, 22). The design of the distal shape of the retractor in (FIGS. 21 and 22) contains a known semicircular shape to grip the shank of a screw and a wider opening to grip the head of a pedicle screw if it is not possible to engage the shank underneath the head (because of bony obstruction) in which case the retractor will still engage the head sufficiently to improve stability over non engaging retractors. Further enhancement has been added by the use of the threaded features.

### Preferred Embodiments

1. A Single retractor blade and flexible loop at a distal end for anchorage around a pedicle screw or other bone anchor (see FIG. 3 for example). A single blade with flexible loop is looped around a pedicle screw before or after insertion. The blade material can be selected from a metals or plastics. Preferably, the material type and gauge allow flexibility so the blade can rotate about a hinge or anchor point. The single blade retractor would typically be used in or after screw insertion from a midline approach where all the tissues to be retracted are in the lateral direction and only one blade is required for each pedicle screw.

A retractor blade has a proximal and distal end. Close to the distal edge of the blade, there is a row of horizontal holes (or openings). There are two holes one above the other in the midline. Running at least part of the length of the blade from proximal to distal, the blade has 1 or more channels as shown in FIGS. 1, 2, 4 and 5. A smooth line (like fishing line, plastics suture material or other suitable flexible material) maybe knotted or otherwise fixed at one end (fixed end) at the top of the blade to prevent it sliding. The other free end is passed down through the vertical channel in the blade and then out one hole and back through another hole in the distal end of the blade thus forming a loop of line adjacent to and in front of the distal edge of the blade—see for instance loop 41 of FIG. 4. The free end then feeds back up to the top of blade thru the same or a second vertical channel. In FIG. 2 the thread passes through the same vertical channel but in FIG. 4 thread 40 passes along lateral channels. At the top of the blade, the free end of the line can be fixed by being pressed into a cut in the plastic retractor blade—see securing notches 25 and 26. The cut may be made in manufacture or with scalpel or scissors in theatre. Alternative methods of fixation of the free end include a separate clip, a ‘bulldog’ clamp applied over line, or a lead ball compressed around line. However achieved tension can be maintained in the line, preventing the line from sliding back down the blade, or the blade sliding up the line.

Insertion and use : With the line loose forming a small horizontal hoop of the required size, the blade and line are placed into wound and hooped over the head of an anchor. By drawing the loose end of the line tight, the distal loop is drawn tight around the neck of the anchor and the blade drawn close to the anchor. The bone around the screw prevents the blade and line from being inserted too distally, the head prevents the threaded loop from coming off proximally. The line is then secured at the top. This tight distal loop engages the anchor and prevents the blade from lifting out of the wound but flexibility, mobility and some elasticity of line allows the retractor blade to move and rotate. As the line is free to travel around the anchor, the blade is still able to rotate axially around the anchor to its preferred position. The relative fixation to the anchor also allows rotation of the proximal blade end moving relative to the relatively fixed distal end for retraction or relaxation of the soft tissues.

To release and remove the retractor blade assembly, the line thread may be cut anywhere and the blade and line then pulled out completely. An alternative to cutting the line is that the free end of the line is released by unclipping it. By pulling the blade a little so the blade slides over the line, the distal loop is loosened allowing the whole assembly to be lifted off its anchor and removed intact. This second method allows blade insertion and removal to be repeated as required.

2. PS retractor (see FIGS. 21,22) This is similar in application to the retractor of 1 but retractor has short legs or features from distal edge that help engage under or around pedicle screw head and guide line into a loop.

3. Wide bladed version with two hoops as in FIG. 9 near either end to enable fixation to two or more pedicle screws. See also (FIG. 15)

4. Dual Bladed threaded retractor. An assembly of two similar blades for Wiltse approach or percutaneous approaches for pedicle screw insertion: Connection of two similar blades is reinforced by use of a thread. (see FIG. 14) In this embodiment the 1st blade is threaded as per the single blade version above but into distal loop a second blade is introduced looping through several of its distal holes only before re-entering the first blade. Drawing the line tight would thus create (with flat blade ends) a rectangular shape. (see FIG. 17). If the distal ends of the blades were shaped accordingly eg semicircular, drawing the line tight would create a round shape. (see FIG. 14).

Various distal blade end shapes are envisaged. Thus paired opposing blades, according to one embodiment, are linked and the connection reinforced by the line to create a device (with the line tight) with tapered distal ends looking very similar to the intact Serengeti retractor. It also differs from the Serengeti retractor because the size of the distal opening in the present invention can be varied almost infinitely by loosening the flexible thread. The dual bladed embodiment can be inserted with the line tight forming an assembly similar in appearance to the Serengeti that can be inserted with the pedicle screw. However unlike the Serengeti, it can also be inserted after screw insertion with the threaded line loose. It differs significantly from the Serengeti because the size of the distal opening in this device can be varied by loosening the nylon. The prior art one piece device (the K2m) is a one piece device and must be inserted while attached to the screw and it cannot be reattached afterwards. The retractor according to the present invention can be inserted with the line tight as per the K2m but unlike the K2m, also after screw insertion with the line loose The K2m requires a special tool to snap the blades apart for removal, whereas the present invention requires only loosening or cutting the line. Such a dual bladed device has applications with any implanted surgical screw or anchor but lends itself particularly to percutaneous insertion techniques.

This anchor(s) can be used to fix one of the blades directly by insertion through a passage in the blade. In such a case the line would function not to grip the anchor directly but to link the non anchored blades to the anchored blade. These blades may also utilize anchor(s) that have features that allow engagement with the line eg a hook, and may also have features that engage with the sides of the blades eg matching convex and concave surfaces between screw head and the edges of the blades. Thus with line drawn tight the blades engage with the sides of the anchors.

The present intention allows the creation of an open space inside the blades that could be enlarged by spreading the threaded blades. Also, unlike where the anchor is used as a fixation point for one of the retractor blades, this arrangement with the anchor securing the line but not directly fixed to the blades has more flexibility in that the blade loop is free to rotate around the anchor utilizing the flexibility and elasticity of the line. It also means the blades and line can be withdrawn from the wound leaving only the anchor and then replaced either to the same location or if using a non circular blade formation by rotating this formation as desired. Rotation of the blades could occur as discussed under Blade edges and Multiple blade devices in section on General description.

7. Two curved end blades. These blades conform to curves of the vertebral bodies in their axial plane as discussed in the applicant's co pending PCT application—application number PCT/AU2009/.

A blade may be detachable or malleable, rotating and other variable engagements are envisaged. Blades may also be separate to the handle to allow fixation of the blade at various angles. Various materials may be used for the retractor blade including metals and plastics and malleable and radiolucent materials. Various sizes and shapes are envisaged.

Variations in the retractor may be made to accommodate different variation in engagement to vertebrae. For instance the length, width and relative heights of horizontal and vertical sections, may vary to accommodate, different locations in the spine and different types of procedures. Also, corners of the device may be radiused and in the lumbar spine build up sections over the corners added in order to retract blood vessels.

It will be further recognised by persons skilled in the art that numerous variations and modifications may be made to the invention without departing from the overall spirit and scope of the invention broadly described herein. Such modifications would allow adaptation of key concepts to provide locking of distraction devices for use in anterior or posterior spinal surgery throughout the length of a spine or in orthopaedics or other surgical disciplines where bony fixation is available.

1. A retractor blade for retracting soft tissues during surgery, the blade comprising:
a blade body having a first proximal end and a second distal end, the first end including means to allow connection of the blade body to a blade support member,
a formation at its distal end which is configured to releasably engage a second co-operating retractor blade; and
means to enable engagement of the retractor blade to an anchor.

2. A retractor blade according to claim 1 wherein, the retractor blade and second co-operating retractor blade releasably engage co-operating connecting formations.

3. A retractor blade according to claim 2 wherein, the formations which enable co-operation of one blade with another blade are opposite gender connections.

4. A retractor blade according to claim 3 wherein, the formations which enable co-operation of one blade with another blade engage in snap fit relationship.

5. A retractor blade according to claim 4 wherein, the formations allow co-operating blades to rotate towards and away from each other.

6. A retractor blade according to claim 5 wherein, the means to enable releasable engagement of the retractor blade with the anchor comprises at least one opening in the blade which receives at least one retaining thread which is fed through said at least one opening to allow releasable engagement via the second end of the retractor blade with the anchor.

7. A retractor blade according to claim 6 wherein, the at least one opening comprises passages which receive the at least one retaining thread.

8. A retractor blade according to claim 7 wherein the passages are arranged to enable the at least one thread to enter or exit the passages

9. A retractor blade according to claim 8 wherein there is one thread provided which co-operates with the blade to form at least one distal end loop which engages the anchor.

10. A retractor blade according to claim 9 wherein, the distal end includes formations which each include openings which receive the at least one thread.

11. A retractor blade according to claim 10 wherein the blade has aligned passages which travel at least part way along of the blade.

12. A retractor blade according to claim 11 wherein the thread extends from the first proximal end to the second distal end and back to the first end.

13. A retractor blade according to claim 12 wherein the distal end includes an entry opening and an exit opening allowing the at least one thread to form a loop at the distal end.

14. A retractor blade according to claim 13 wherein the passages in the blade are arranged to allow a thread to be fed into an opening in the proximal end to exit through one opening in the distal end.

15. A retractor blade according to claim 14 further comprising another opening in the distal end which receives an end of the at least one thread and another opening at the proximal end from which the at least one thread exits.

16. A retractor blade according to claim 15 wherein the openings in the proximal end accommodate free ends of the at least one thread.

17. A retractor blade according to claim 16 wherein the free ends of the at least one thread allow a surgeon to control the action of the at least one thread for the purpose of engagement and disengagement.

18. A retractor blade according to claim 17 wherein the passages are disposed along the blade at least part way along the length of a blade.

19. A retractor blade according to claim 18 wherein the passages are cannulated and are disposed along an edge or a face of each retractor.

20. A retractor blade according to claim 19 wherein the anchor is a bone screw.

21. A retractor blade according to claim 20 wherein the anchor is a second retractor blade.

22. A retractor blade assembly for retraction of soft tissue, the blade assembly comprising:
a pair of co-operating blades each having a generally elongated blade body having first and second ends, the first end including means allowing connection of the blade body to a support member, the second end comprising means to enable anchorage of the blade, wherein said means to connect the blade body at its distal end comprises a releasable coupling which allows one blade to rotate relative to the other blade.

23. A retractor blade assembly according to claim 22 further comprising a flexible thread capable of forming a connecting loop to reinforce the connection between the co-operating blades.

24. A retractor blade assembly according to claim 23 wherein the co-operating blades can move along the thread so that the position of each blade is adjustable but mutually retained.

25. A retractor blade assembly for retraction of soft tissue during surgery, the blade assembly comprising: at least one generally elongated blade having first and second ends, a thread which passes through openings in the blade to form at least one loop of thread at the second end.

26. A retractor blade assembly according to claim 25 wherein the thread is configured to engage an anchor.

27. A retractor blade assembly according to claim 26 wherein, free ends of the thread exit the blades via openings in the proximal end of the blades thereby enabling a surgeon to tension the closed loop.

28. A retractor blade assembly according to claim 27 wherein the loop at the second end of the blade is configured to engage a bone anchor or a corresponding distal end of a co-operating retractor blade.

29. A retractor blade assembly according to claim 6 wherein the thread consists of a material is-selected from the group consisting of flexible metal or plastics.

30. A retractor blade assembly according to claim 29 wherein the thread is a plastics suture material.

31. A retractor blade assembly according to claim 29 wherein the thread is a flexible wire.

32. A retractor blade according to claim 1 wherein the blade comprises a material selected from one of the group consisting of flexible plastics, rigid plastics or a metal.

33. A retractor blade assembly according to claim 26 wherein, the anchor is a pedicle screw.

34. A retractor blade for retraction of soft tissue the blade comprising: a generally elongated blade body having first and second ends, the first end providing means for connection of the blade body to a support member, the second end including means to enable the retractor to engage an anchor via a co-operating retaining thread.
